(19) United States
(12) Plant Patent Application Publication
(10) Pub. No.: US 2008/0155721 P1
(43) Pub. Date: Jun. 26, 2008

(54) Kiwi plant named 'Hongyang'

(76) Inventors: Li Mingzhang, Beijing (CN); Wang Mingzhong, Beijing (CN)

Correspondence Address: STRATTON BALLEW, 213 S 12TH AVE, YAKIMA, WA, 98902, US

(21) Appl. No.: 11/645,130

(22) Filed: Dec. 22, 2006

(51) Int. Cl. A01H 5/00

(52) U.S. Cl. PLT/156

(57) ABSTRACT

A new cultivar of kiwi plant (Actinidia chinensis) named ‘Hongyang’ is disclosed. The fruit of ‘Hongyang’ is notable for its green and reddish-purple flesh.

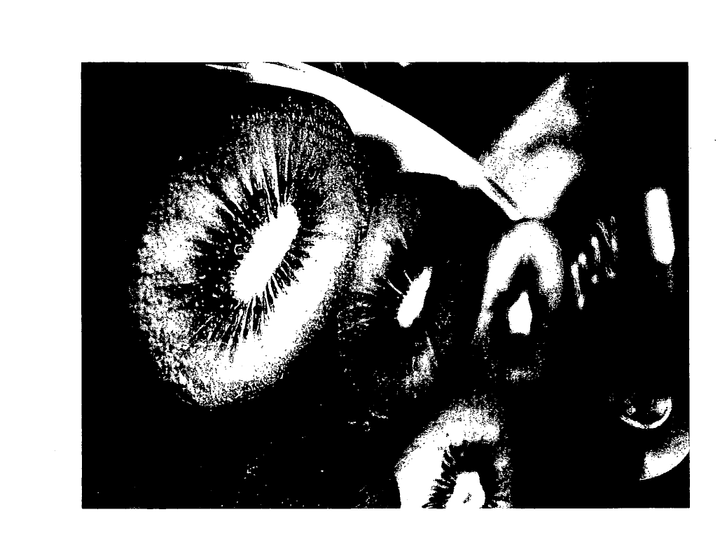

### CROSS REFERENCE TO RELATED APPLICATIONS

None.

### STATEMENT REGARDING FEDERALLY SPONSORED RESEARCH OR DEVELOPMENT

None.

### LATIN NAME OF THE GENUS AND SPECIES OF THE PLANT CLAIMED

Actinidia chinensis

### VARIETY DENOMINATION

‘Hongyang’

### BACKGROUND OF THE INVENTION

A new and distinct kiwi plant (Actinidia chinensis) named ‘Hongyang’ is described herein. ‘Hongyang’ originated as a seedling of Actinidia chinensis var. rufopulpa. In 1984, the inventors of the claimed plant sowed seeds of wild Actinidia chinensis var. rufopulpa at Chengdu, Sichuan Province, China From the resulting seedlings, 3213 healthy young plants were selected for observation. Of the selected plants, 921 set fruit and developed seeds. ‘Hongyang’ was among the 921 plants, and was notable for its unique red-cored flesh. From 1987 to 2000, ‘Hongyang’ was asexually propagated by grafting at Chengdu and other locations, and has been found to remain true to type through successive asexually propagated generations.

### BRIEF DESCRIPTION OF THE INVENTION

‘Hongyang’ was selected for unique flesh coloration. The flesh surrounding the seeds near the core of the fruit is red, while the remainder of the flesh is green in color. ‘Hongyang’ is distinguishable from its parent Actinidia chinensis var. rufopulpa by this and other characteristics, as noted in the table below:

TABLE 1
Comparison of ‘Hongyang’ to Parent Actinidia chinensis
var. rufopulpa (measurements given are approximate)

| | | Actinidia chinensis |
|---|---|---|
| | ‘Hongyang’ | var. rufopulpa |
| Fruit: Shape of Top | Concave | Flat |
| Fruit: Color of Skin | Greenish- | Yellowish brown |
| | brown | |
| Fruit: Color of Core | Red purple | Greenish white |

### BRIEF DESCRIPTION OF THE DRAWING

The FIGURE shows sectioned fruit of ‘Hongyang’ kiwi.

### DETAILED BOTANICAL DESCRIPTION

The following detailed botanical description is based on observations made during the 2003 growing season at Chengdu, Sichuan Province, China. All colors are described according to The Royal Horticultural Society Colour Chart. It should be understood that the characteristics described will vary somewhat depending upon cultural practices and climatic conditions, and will vary with location and season. Quantified measurements are expressed as an average of measurements taken from a number of individual plants of the new variety. The measurements of any individual plant, or any group of plants, of the new variety may vary from the stated average.
Plant: sex — female
Plant: vigor — medium (similar to ‘Hayward’)
Young shoot: hairiness — present
Young shoot: length of hair — short
Young shoot: thickness — medium (0.75 to 0.80 cm)
Young shoot: color — light brown
Stem: number of lenticels — few (avg. 7.9)
Stem: shape of lenticels — ellipsoid
Stem: size of bud support — medium (0.46 to 0.55 cm)
Stem: leaf scar — shallow (1.3 to 2.3 mm)
Young leaf: shape — cordate
Young leaf: shape of apex — obtuse
Young leaf: arrangement of basal lobes — slightly apart
Young leaf: serrate margin — few
Young leaf: anthocyanin coloration of petiole — strong
Leaf blade: area — small (avg. less than 180 cm²)
Leaf blade: undulation on upper side — strong
Leaf blade: green color of upper side — dark
Leaf blade: length of petiole — short (avg. less than 9.0 cm)
Flower: number of inflorescences — few (avg. less than 3.5)
Flower: number of lateral florets — few (avg. Less than 0.4 per lateral)
Flower: size — small (avg. less than 40 mm)
Petal: length/width ratio — small (avg. less than 1.15)
Petal: main color on adaxial side — white
Petal: curvature of apex — cup shaped
Petal: number — medium (avg. 6.6 to 7.5 per flower)
Style: number — medium (30 to 39.9 per flower)
Style: attitude — erect
Ovary: shape — ellipsoid
Ovary: anthocyanin coloration of longitudinal profile — present
Anther: number — medium (avg. 180.0 to 249.9 per flower)
Fruit: general shape — cylindrical
Fruit: weight — avg. 70 to 80 g
Fruit: position of the largest cross section — fruit middle
Fruit: persistence of sepals — absent
Fruit: shape of top — concave
Fruit: color of skin — greenish brown
Fruit: color of flesh — mostly green, reddish-purple near center
Fruit: length of hair — short
Fruit: adherence of hairs to skin — weak
Fruit: length of stalk — short (avg. 2.5 to 3.4 cm)
Fruit: thickness of stalk — thin (avg. less than 2.4 mm)
Fruit: ratio of stalk/fruit length — small (avg. 0.50 to 0.59)
Fruit: color of inner pericarp (around seeds in flesh) — red
Fruit: general shape of core (in cross section) — ellipsoid
Flesh: percent soluble solids — high (avg. greater than 16%)
Fragrance — heavy
Fruit: sweetness — high
Time of vegetative bud burst: — very early
Time of beginning of flowering: — early
Time of maturity for harvest: — very early
Time of beginning of fruiting: — very early
Difficulty of post maturity: — easy
Shelf life: — medium

We claim:

1. A new and distinct kiwi plant substantially as shown and described herein.
